(19) United States
(12) Patent Application Publication
(10) Pub. No.: US 2022/0013031 A1
(43) Pub. Date: Jan. 13, 2022

(54) DEMENTIA PATIENT TRAINING SYSTEM USING VIRTUAL REALITY

(71) Applicant: C2 MONSTER CO., LTD., Seoul (KR)

(72) Inventor: Jin Sung CHOI, Uiwang-si, Gyeonggi-do (KR)

(73) Assignee: C2 MONSTER CO., LTD., Seoul (KR)

(21) Appl. No.: 17/311,989

(22) PCT Filed: Nov. 25, 2019
(86) PCT No.: PCT/KR2019/016232

§ 371 Date: Jun. 8, 2021

(30) Foreign Application Priority Data
Dec. 10, 2018 (KR) 10-2018-0157975

(51) Int. Cl. G09B 9/00; A61B 5/00; A61B 5/11; G16H 20/70; G16H 50/30

(52) U.S. Cl. CPC G09B 9/00; A61B 5/4088; G16H 50/30; G16H 20/70; A61B 5/11

(57) ABSTRACT

Disclosed is a dementia patient training system using virtual reality (VR).

### TECHNICAL FIELD

The present disclosure relates to a dementia patient training system using virtual reality (VR).

### BACKGROUND ART

With the development of a computer technology, VR technology has also developed significantly, and has been actually applied to various fields. Nowadays, fields, to which the VR technology is applied, such as education, shopping, and the like are being gradually expanded beyond a game field and an entertainment field. Accordingly, a demand on cognitive ability improvement mission is also increasing.

In the meantime, as the aging society in Korea currently is progressing, silver industry, which is an industry for the elderly, is being developed in various fields. The silver industry is being developed in various fields such as a home care service, an intermediate care facility business, a nursing home and nursing facility, a medical service industry, and procedure operation business of care housing.

Dementia refers to a brain disease that the elderly suffer a lot. The dementia indicates a condition in which a person who has been living normally has suffered a significant disruption in daily life due to continuous and overall decline in cognitive function as the brain function of the person is damaged by various causes.

The medical service industry for healing dementia is developed at a therapeutic level.

### DETAILED DESCRIPTION OF THE INVENTION

### Technical Problem

The present disclosure produces a dementia patient training system using VR that is capable of inducing interest and continuously and repeatedly providing training, by providing a dementia patient with a cognitive ability improvement mission in a virtual environment.

In the meantime, the technical problems to be solved by embodiments of the present disclosure are not limited to the aforementioned problems, and other technical problems that are not mentioned will be clearly understood by those skilled in the art from the following description.

### Technical Solution

According to an embodiment of the present disclosure, a dementia patient training system using virtual reality (VR), which includes a server providing the VR to a dementia patient accessed over a network may include a process module improving a virtual environment of the VR and providing the patient with at least one mission content for improving a plurality of cognitive abilities in the virtual environment, a storage module storing the mission content, selection data of the patient, motion data of the patient, and data about whether a mission of the patient is successful, a communication module communicating with a terminal of the patient over the network, and a cognitive ability determination module determining a cognitive ability of the patient through the selection data of the patient and the motion data of the patient for a mission provided by the process module. The communication module may receive the selection data of the patient and the motion data of the patient. The process module may reflect the selection data of the patient and the motion data of the patient to the virtual environment.

The mission content in the storage module may be classified into a memory group for verifying a memory cognitive ability, a concentration group for verifying a concentration cognitive ability, a calculation ability group for verifying a calculation cognitive ability, a space-time group for verifying a spatiotemporal cognitive ability, a thought processing group for verifying a thought processing cognitive ability, and a language improvement group for verifying a language cognitive ability. The process module may select at least one mission from each of the memory group, the concentration group, the calculation ability group, the space-time group, the thought processing group, and the language group so as to be provided to the patient.

Difficulty levels of a plurality of missions stored in each of the memory group, the concentration group, the calculation ability group, the space-time group, the thought processing group, and the language group in the storage module may be set in a grade order.

The process module may select a mission of a grade corresponding to the cognitive ability of the patient in each group, a mission of a grade immediately below the grade corresponding to the cognitive ability of the patient, and a mission of a grade immediately above the grade corresponding to the cognitive ability of the patient, which are 1:1:1, based on the cognitive ability of the patient for each group, which is classified at a previous try, so as to be provided to the patient at a next try. When it is determined, based on the cognitive ability of the patient for the each group classified at the previous try, that the cognitive ability of the patient in a specific group is at the highest grade, the process module may select a mission of a grade corresponding to the cognitive ability of the patient and a mission of a grade immediately below the grade corresponding to the patient's cognitive ability, which are 2:1, so as to be provided to the patient at the next try. When it is determined, based on the cognitive ability of the patient for the each group classified at the previous try, that the cognitive ability of the patient in a specific group is at the lowest grade, the process module may select a mission of a grade corresponding to the cognitive ability of the patient and a mission of a grade immediately above the grade corresponding to the patient's cognitive ability, which are 2:1, so as to be provided to the patient at the next try.

The cognitive ability determination module may store the cognitive ability of the patient for the each group, which is classified at each repeated try, in the storage module and may determine whether the cognitive ability of the patient is improved or declined, based on information about the cognitive ability of the patient that accumulated at the each repeated try.

The process module may increase a time distribution amount within a predetermined time for a specific group, in which a cognitive ability declines, and may decrease a time distribution amount within the predetermined time for a specific group in which a cognitive ability is improved, based on the cognitive ability of the patient for each group classified at the previous try.

The communication module may transmit the information about whether the cognitive ability of the patient in the each group is improved or declined, to the outside at a predetermined period or at an external request.

A plurality of patients may be capable of accessing the server through terminals of the plurality of patients. The plurality of patients may include a first patient and a second patient. The process module may provide the plurality of patients with mission content in which the first patient and the second patient collaborate or compete with each other.

The second patient may access the server through a terminal of the second patient. When the second patient accepts a friend invitation event of the first patient in a virtual environment of the second patient, the process module may receive a virtual environment of the first patient.

### Advantageous Effects of the Invention

According to an embodiment of the present disclosure, it is possible to induce interest and to continuously and repeatedly provide training, by providing a dementia patient with a cognitive ability improvement mission in a virtual environment.

In the meantime, effects obtained in the present disclosure are not limited to the above-mentioned effects, and other effects that are not mentioned will be clearly understood by those skilled in the art from the following description.

### DESCRIPTION OF THE DRAWINGS

FIG. 1 is a configuration diagram illustrating a configuration of a dementia patient training system using VR, according to an embodiment of the present disclosure.

FIG. 2 is a block diagram illustrating a configuration of a server in FIG. 1.

FIG. 3 is a flowchart of a dementia patient training method using VR, according to an embodiment of the present disclosure.

FIGS. 4 to 23 are screens of a virtual environment illustrating a mission provided by a dementia patient training system using VR and a process in which a patient resolves the mission.

### BEST MODE

Embodiments of the present disclosure are manufactured to more completely describe the present disclosure to those skilled in the art. The following embodiments may be modified in many different forms, and the scope of the present disclosure is not limited to the following embodiments. Accordingly, these embodiments are produced to make the present disclosure more faithful and complete, and to completely deliver the spirit of the present disclosure to those skilled in the art.

In addition, in the following drawings, each configuration is exaggerated for convenience and clarity of description, and the same reference numerals in the drawings refer to the same elements. As used in this specification, a term “and/or” includes one of corresponding listed items and all combinations of one or more of the corresponding listed items.

Terms used in this specification are provided to describe a specific embodiment, but not to limit the present disclosure.

As used in this specification, a singular form may include a plural form unless contexts clearly indicate another case. Besides, when used in this specification, “comprise” and/or “comprising” does not exclude presence or addition of one or more other shapes, numbers, actions, members, elements, and/or groups in addition to the aforementioned shapes, numbers, steps, actions, members, elements and/or groups thereof.

Hereinafter, embodiments of the present disclosure will be described in detail with reference to the accompanying drawings.

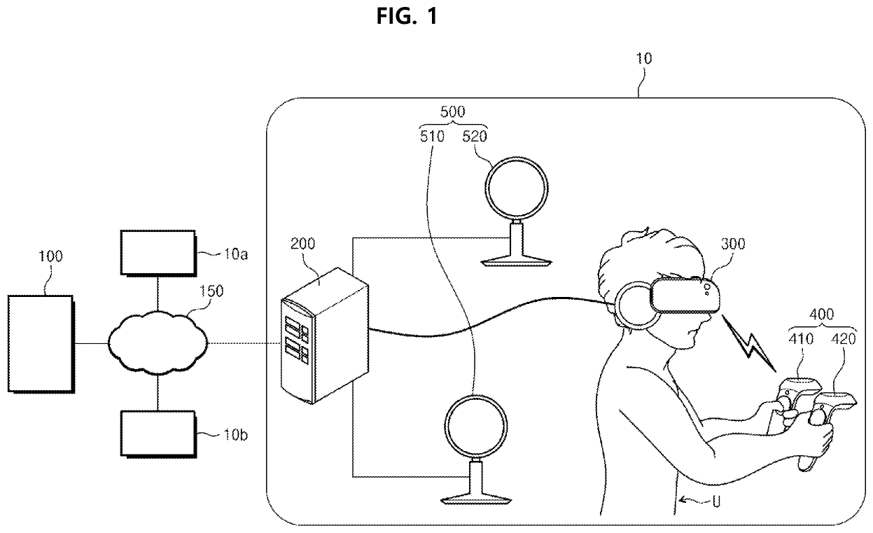

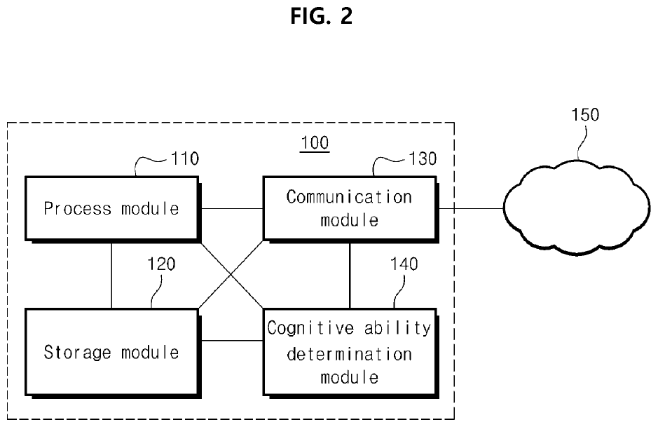

FIG. 1 is a configuration diagram illustrating a configuration of a dementia patient training system using VR, according to an embodiment of the present disclosure. FIG. 2 is a block diagram illustrating a configuration of a server in FIG. 1.

Above all, referring to FIGS. 1 and 2, a dementia patient training system using VR includes a server 100 and user environments (10, 10a, 10b).

The user environment 10 may include a terminal 200, an output unit 300, an input unit 400, and a sensing unit 500.

The server 100 may refer to a central system that manages the accessed terminal 200 and a VR image. The server 100 may manage users through a network 150, may manage a user connection status, and may generate a virtual environment screen of VR through base data of the terminal 200 of a user so as to be provided to the output unit 300 of the user.

Also, the server 100 may provide a market that provides product information associated with cognitive ability improvement, or may provide habit information associated with cognitive ability improvement.

For example, the server 100 may provide users (patients) with the habit information associated with cognitive ability improvement such as meditation, reading, and tea drinking habits or the product information associated with cognitive ability improvement such as tea, food, and supplements.

Herein, the habit information associated with cognitive ability improvement may include specific papers or article links. The product information associated with cognitive ability improvement may include product sale links.

In this way, the users (patients) may receive repetitive and continuous dementia-related information, thereby improving cognitive ability, alertness, or perseverance.

Furthermore, an operation manager may provide a product link to the users (patients), thereby obtaining additional advertising revenue.

In the meantime, the server 100 may basically provide a virtual environment that a user is capable of experiencing in a first-person view that is viewed from the user's character. In addition, the server 100 may provide a viewpoint change environment, in which the user's character and an invited opponent character are capable of being viewed together in a third-person view at a specific point, by changing the viewpoint.

The server 100 may include a process module 110, a storage module 120, a communication module 130, and a cognitive ability determination module 140.

For example, the process module 110 may include a processor or a micro controller unit (MCU).

The process module 110 may implement a virtual environment of VR, may provide cognitive ability improvement mission content (or mission content) to a user (a dementia patient) in a virtual environment, and may reflect the user's selection data and motion data received through the communication module 130 to the virtual environment.

Herein, missions provided in a virtual environment provided by the process module 110 may include a mission for adapting VR, a mission for verifying and improving a memory cognitive ability, a mission for verifying and improving a concentration cognitive ability, a mission for verifying and improving a calculation cognitive ability, a mission for verifying and improving a spatiotemporal cognitive ability, a mission for verifying and improving a thought processing cognitive ability, and a mission for verifying and improving a language cognitive ability.

In an embodiment of the present disclosure, the cognitive ability improvement mission content may require changes in motion according to a location of the user's character, a motion of the user's character, a movement of an object, and a placement of the object in a virtual environment, and may include data in various types of photos or images, or a program that allows the user to enjoy and experience a virtual space embodied in 3D in a 360-degree direction in a first-person view or a third-person view.

Moreover, the cognitive ability improvement mission content may require a process of resolving a mission through changes in the location, the motion, a movement of the object, and a placement of the object, in a situation in which a plurality of users collaborate or compete with each other in a virtual space.

In the meantime, the cognitive ability improvement mission content may be captured based on field of view of the user's specific character, and may be played in the user's first-person view or third-person view during playback.

The process module 110 may provide a list screen to a user (U) in the cognitive ability improvement mission content. Herein, the list screen may include a friend list and a mission-type content list.

The stored at least one friend may be displayed in the friend list together with a character thumbnail, and online or offline information of the corresponding friend may be displayed in the friend list.

The mission-type content list may be classified into an adaptation group for adapting VR, a memory group for verifying the memory cognitive ability, a concentration group for verifying the concentration cognitive ability, a calculation group for verifying the calculation cognitive ability, a space-time group for verifying the spatiotemporal cognitive ability, a thought processing group for verifying the thought processing cognitive ability, and a language improvement group for verifying the language cognitive ability.

The list screen may be displayed on a user interface (UI) for a user in a virtual environment.

The storage module 120 may store user data based on login information of the user (U). The user data may include friend data and data obtained by performing a mission at a previous try.

The friend data may include a friend's name, the friend's representative character image, the friend's login information, or the like.

The data obtained by performing the mission at the previous try may cumulatively include information about a result performed by a user on mission content provided in a virtual environment at the previous try, whether the mission content is performed successfully, or the like.

The storage module 120 may store a program for the processing and control of the process module 110, and may perform a function for temporarily storing input/output data. The storage module 120 may also store the usage frequency of each data.

Herein, the storage module 120 may store a mission or information provided through the process module 110, a process of performing a mission of a user (patient), and the presence or absence of a correct answer.

In the meantime, the mission stored in the storage module 120 may be classified into an adaptation group for adapting VR, a memory group for verifying the memory cognitive ability, a concentration group for verifying the concentration cognitive ability, a calculation group for verifying the calculation cognitive ability, a space-time group for verifying the spatiotemporal cognitive ability, a thought processing group for verifying the thought processing cognitive ability, and a language improvement group for verifying the language cognitive ability.

Herein, difficulty levels of a plurality of missions stored in each of the adaptation group for adapting VR, the memory group, the concentration group, the calculation ability group, the space-time group, the thought processing group, and the language group may be set in a grade order.

For example, difficulty levels of the plurality of missions classified into each group may be set to level 1, level 2, level 3, level 4, and level 5.

Herein, as a level is low, the difficulty level may be low. As a level is high, the difficulty level may be high.

In addition, the storage module 120 may cumulatively store a mission execution process of a user (patient) and the presence or absence of a correct answer for each mission try provided daily.

In the meantime, the process module 110 may select at least one mission for each group from among missions, which are classified into a memory group, a concentration group, a calculation ability group, a space-time group, a thought processing group, and a language group stored in the storage module 120, so as to be provided in a virtual environment.

Herein, the process module 110 may appropriately combine missions of level 1, level 2, level 3, level 4, and level 5 for each mission of each group at the first try (the first day) so as to be provided to a user (patient). The cognitive ability determination module 140 may determine the cognitive ability of a user (patient) in each group based on the presence or absence of a correct answer of the user (patient) for a mission at each level so as to be classified for each grade.

For example, when a user (patient) who has received a mission has recorded a correct answer rate of about 70% or more for missions of level 1, level 2, and level 3 at the first try (the first day) in the mission of the memory group and has recorded the correct answer rate of about 40% or less for missions of level 4 and level 5, the cognitive ability determination module 140 may determine that the memory cognitive ability of the user (patient) is at level 3.

Afterward, the process module 110 may select a mission of a grade corresponding to a patient's cognitive ability in each group, a mission of a grade immediately below the grade corresponding to the patient's cognitive ability, and a mission of a grade immediately above the grade corresponding to the patient's cognitive ability, which are 1:1:1, based on the cognitive ability of the user (patient) for each group, which is classified at a previous try, and may provide the selected missions in a virtual environment at a next try.

For example, the process module 110 may select a mission of level 2, a mission of level 3, and a mission of level 4, which are 1:1:1, from missions of the memory group at the second try (next day), and may provide the selected missions to a user (patient), who is determined to have a memory cognitive ability of level 3 at the first try (the first day), in a virtual environment.

In the meantime, when it is determined based on the patient's cognitive ability for each group classified at the previous try that the patient's cognitive ability in a specific group is at the highest grade, the process module 110 may select a mission of the grade corresponding to the patient's cognitive ability and a mission of a grade immediately below the grade corresponding to the patient's cognitive ability, which are 2:1, so as to be delivered to the output unit 300 at the next try. When it is determined based on the patient's cognitive ability for each group classified at the previous try that the patient's cognitive ability in a specific group is at the lowest grade, the process module 110 may select a mission of the grade corresponding to the patient's cognitive ability and a mission of a grade immediately above the grade corresponding to the patient's cognitive ability, which are 2:1, so as to be provided in the virtual environment at the next try.

For example, for a user (patient) determined to have a concentration cognitive ability of level 5 (the highest level) at the first try (the first day), the process module 110 may select a mission of level 5 and a mission of level 4, which are 2:1, in the concentration group mission at the second try (next day) so as to be delivered to the output unit 300. For a user (patient) who is determined to have a calculation cognitive ability of level 1 (the lowest level) at the first try (the first day), the process module 110 may select a mission of level 1 and a mission of level 2, which are 2:1, in the mission of the calculation ability group at the second try (next day) so as to be provided in a virtual environment.

As a result, the ability for determining the cognitive ability of the user (patient) determined by the cognitive ability determination module 140 may be improved.

Afterward, while the above-described process is daily repeated through the cognitive ability determination module 140 and the process module 110, changes in each grade and a correct answer rate of the user (patient) in each grade may be cumulatively stored in the storage module 120.

In the meantime, on the basis of the patient's cognitive ability for each group classified at the previous try, the process module 110 may increase a time distribution amount within a predetermined time for a specific group, in which the cognitive ability of a patient declines, and may decrease a time distribution amount within the predetermined time for a specific group in which the cognitive ability of a patient is improved.

For example, for a user (patient) who is determined to have a concentration cognitive ability of level 5 and a calculation cognitive ability of level 1 through the cognitive ability determination module 140 at the previous try, the process module 110 may relatively increase a mission provision time of the calculation ability group and may relatively decrease a mission provision time of the concentration group at the next try.

In this way, the user (patient) may increase the training time for a relatively-decreased cognitive ability. The user (patient) may increase the amount of activity in one area or another area of a brain for improving a relatively-decreased specific cognitive ability. The user (patient) may activate not only a specific area of the brain that is in charge of the corresponding cognitive ability, but also an area other than the specific area through repetitive training and weight training. Accordingly, the user (patient) may improve the corresponding cognitive ability.

The terminal 200 is a computing device including a processor and a memory, and may be, for example, a mobile terminal such as a tablet PC.

Meanwhile, the terminal 200 is not limited to a mobile terminal, and may be any computer (e.g., a personal computer) as long as the computer includes a processor and a memory.

Also, although not illustrated, the terminal 200 may include an input unit for inputting information, an output unit for outputting the information, a storage unit for storing the information, and a communication unit for transmitting the information to the outside.

The input unit may be an input device that receives data, and may include, for example, a touch pad, a keyboard, a mouse, and a touch pen.

The output unit may be a display device that displays data on a screen, and may include, for example, an LCD or the like.

In the meantime, when the input unit is a touch pad and the touch pad is coupled to the output unit, the input unit and output unit may constitute a touch screen.

The storage unit may be a storage device that stores data such as a file or a data processing program and may include, for example, HDD, SSD, RAM, ROM, a cache, a register, and the like.

Herein, information such as base data associated with VR, cognitive ability improvement mission content executed in a virtual environment of VR, and the executed result may be stored in the terminal 200.

Meanwhile, the terminal 200 may provide the output unit with a login screen for access authentication of the server 100.

The output unit 300 may play a virtual environment screen of VR received from the server 100 so as to be provided to a user wearing a head-mounted device (HMD).

The output unit 300 may output a digital signal of the virtual environment screen of VR received from the server 100 as video and audio.

In the meantime, the output unit 300 is preferably implemented as a HMD. The output unit 300 may include a display for outputting an image and a headset for outputting audio sound.

The input unit 400 may be a controller used in a general VR device. For example, the input unit 400 may be a wired or wireless device, which generates an operation signal for instructing a specific motion of an avatar displayed in VR, such as a game pad, Vive Controller in HTC, TacticalHaptics controller, or the like.

For example, the input unit 400 may include a grip part that is gripped by a user's hand. A hollow may be formed above the grip part. Also, the input unit 400 may include a built-in battery for power supply.

The input unit 400 may include a first input unit 410 and a second input unit 420. The first input unit 410 may receive an operation signal by the user's left hand. The second input unit 420 may receive an operation signal by the user's right hand.

Herein, the operation signal may include a home movement signal, a VR video recording signal, a list screen call signal, a selection signal, and a point movement signal.

For example, the home movement signal, the VR video recording signal, the list screen call signal, or the selection signal may be received from the first input unit 410. The point movement signal may be received from the second input unit 420. However, information about signals received from each of the first input unit 410 and the second input unit 420 is not limited in an embodiment of the present disclosure.

The sensing unit 500 may include a plurality of motion sensors 510 and 520 (e.g., a camera) and a plurality of sensors (not shown) mounted on the input unit 400.

The sensing unit 500 may detect user sensing data according to the user's motion through the plurality of motion sensors 510 and 520 and the plurality of sensors (not shown) mounted on the input unit 400.

The user sensing data may include head tracking data of a user, movement data of an object in a virtual environment, and motion data of a user.

The head tracking data may be data regarding the user's head motion and may be used to determine field of view.

The movement data of an object in a virtual environment may be obtained by movement by a user's walking, manipulation of an input device, or the like.

Besides, the user's motion data may be obtained by a camera or a gesture sensor.

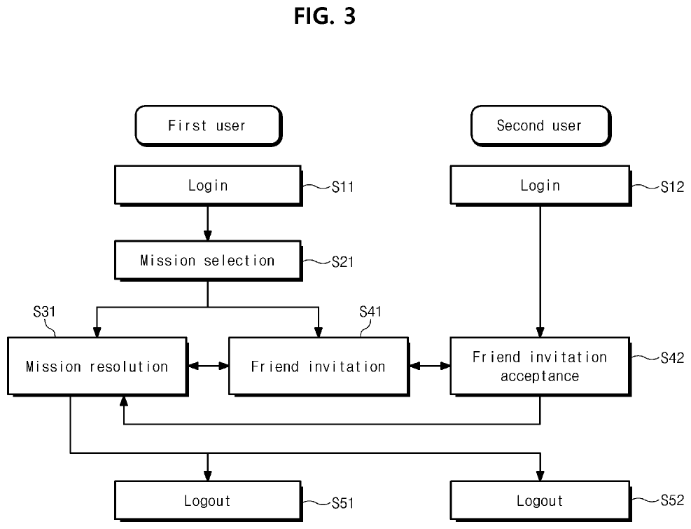

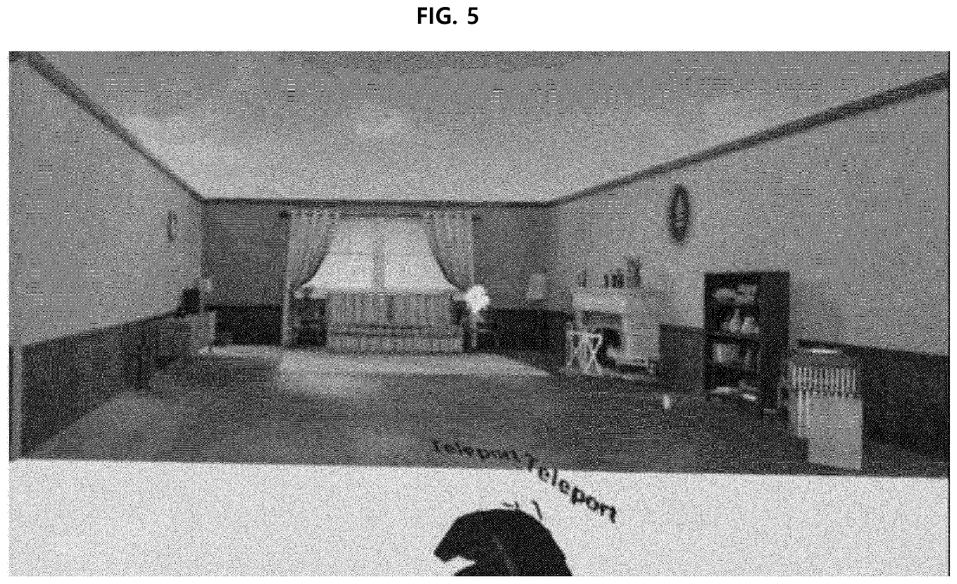

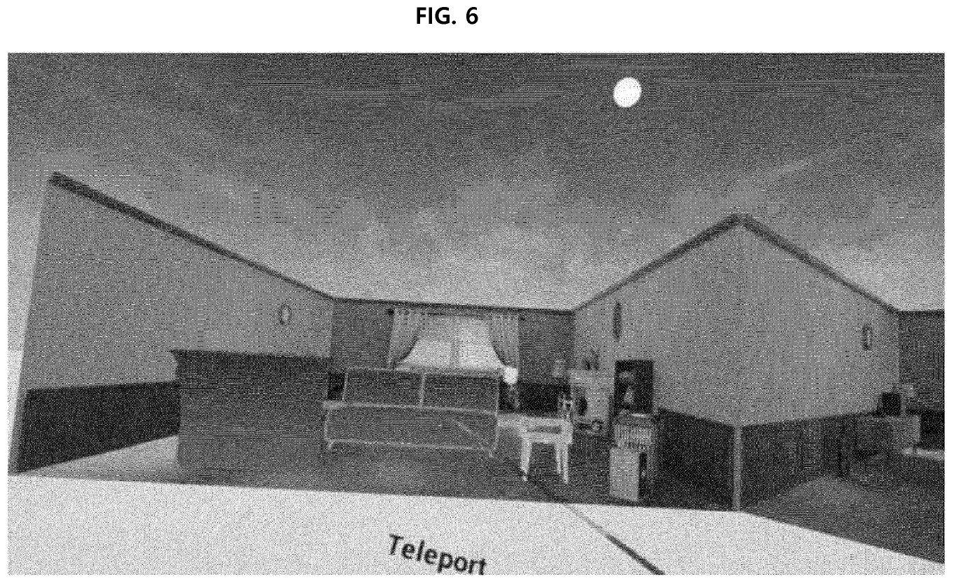

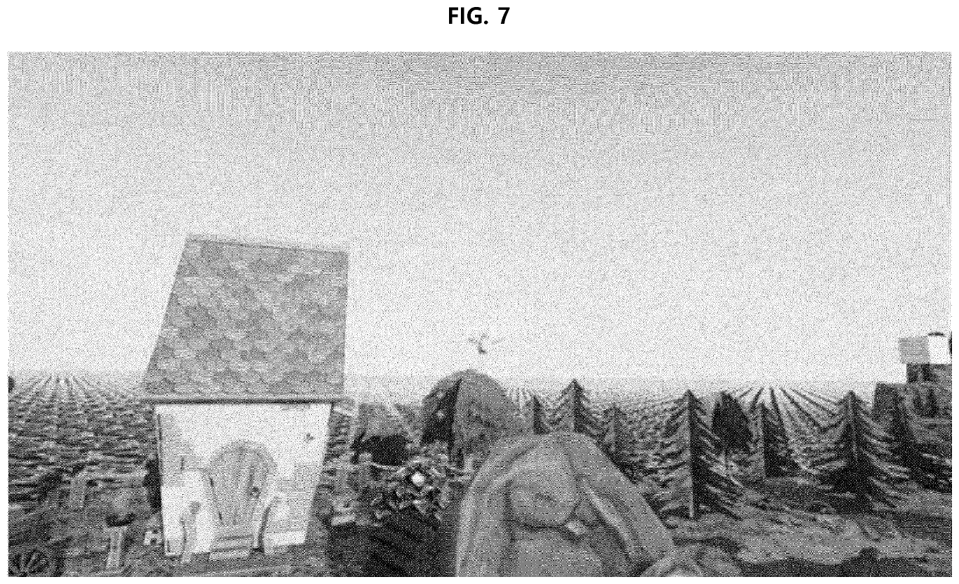

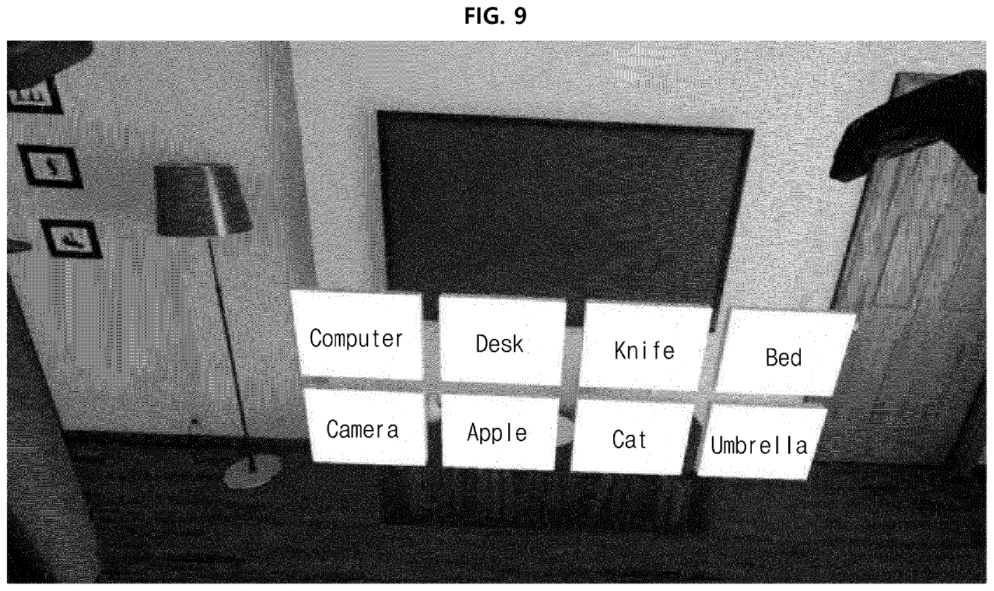

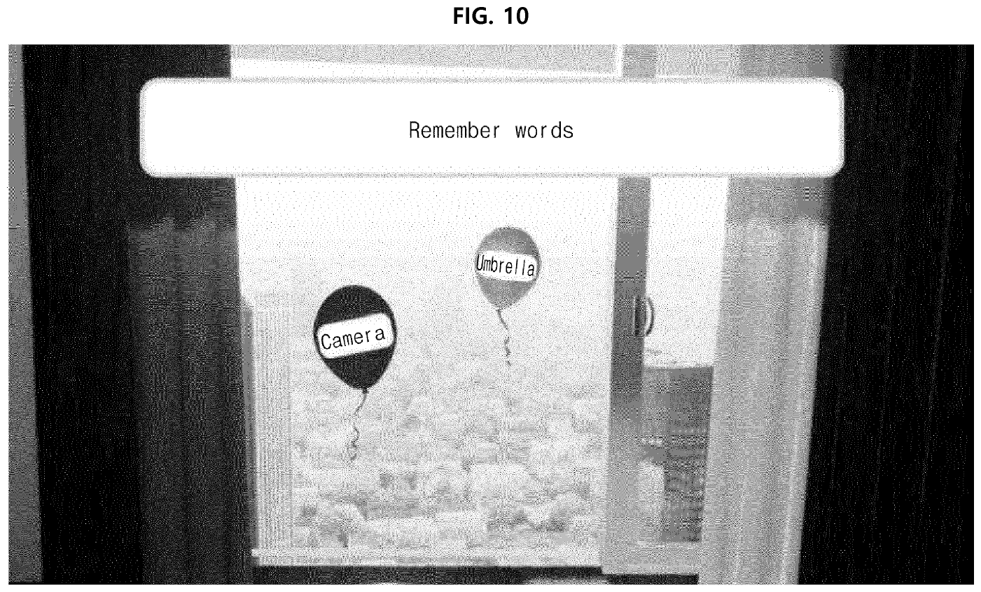

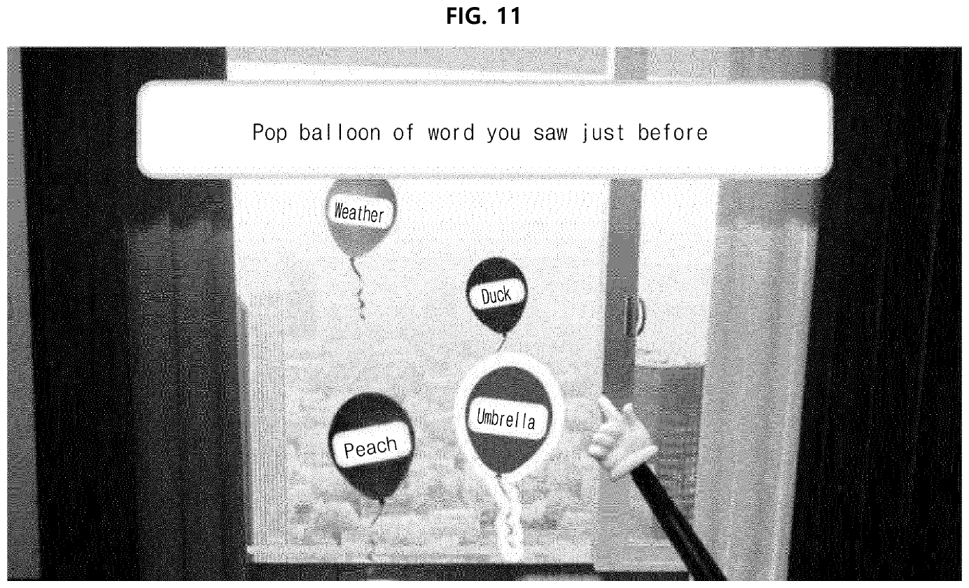

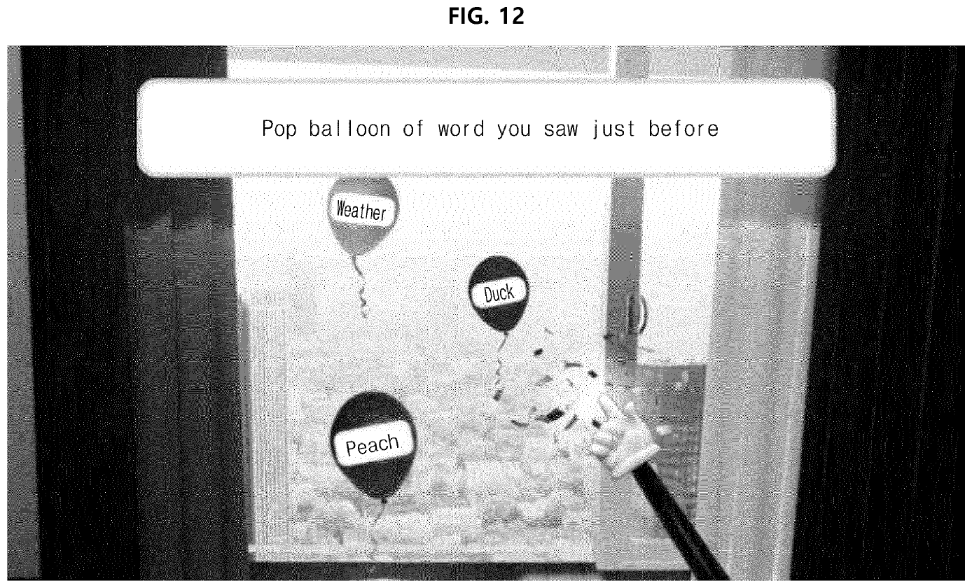

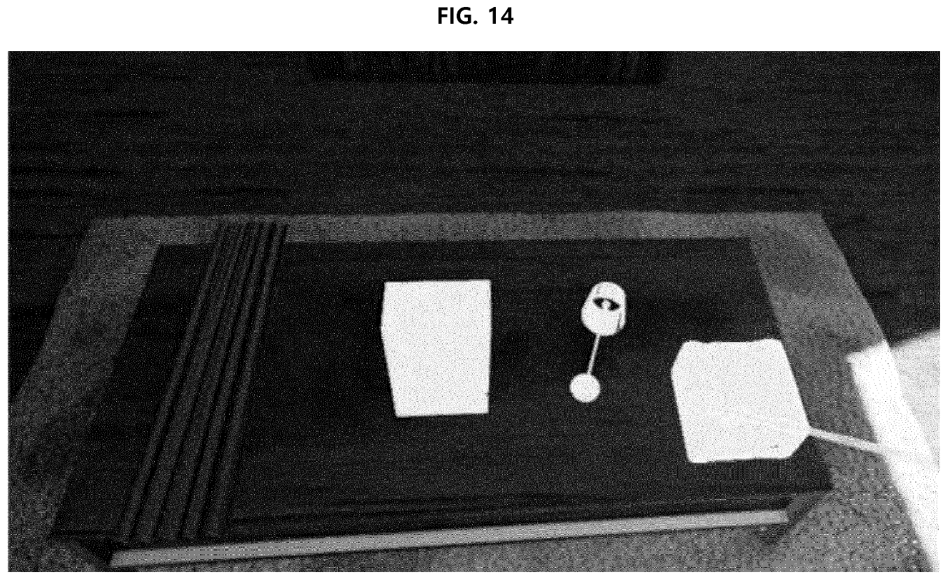

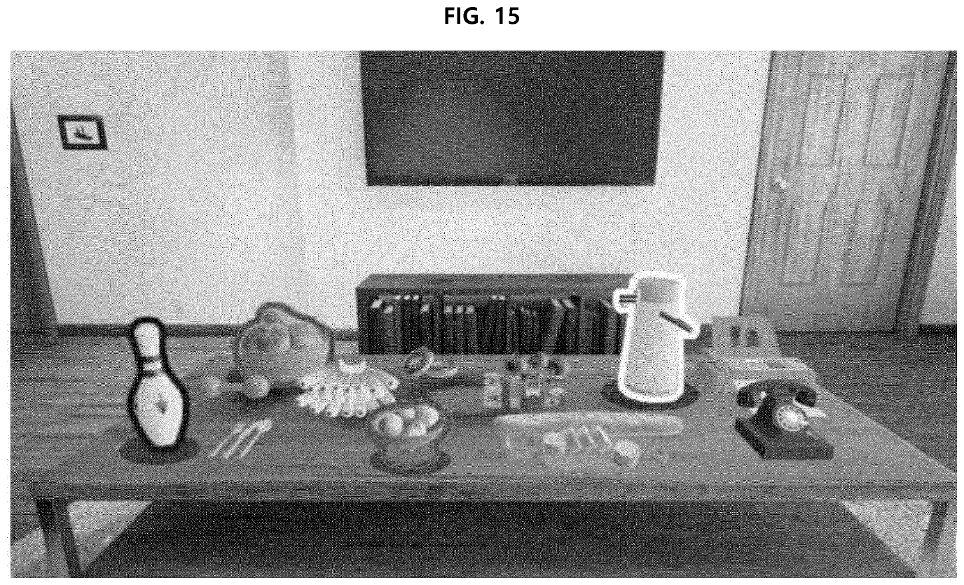

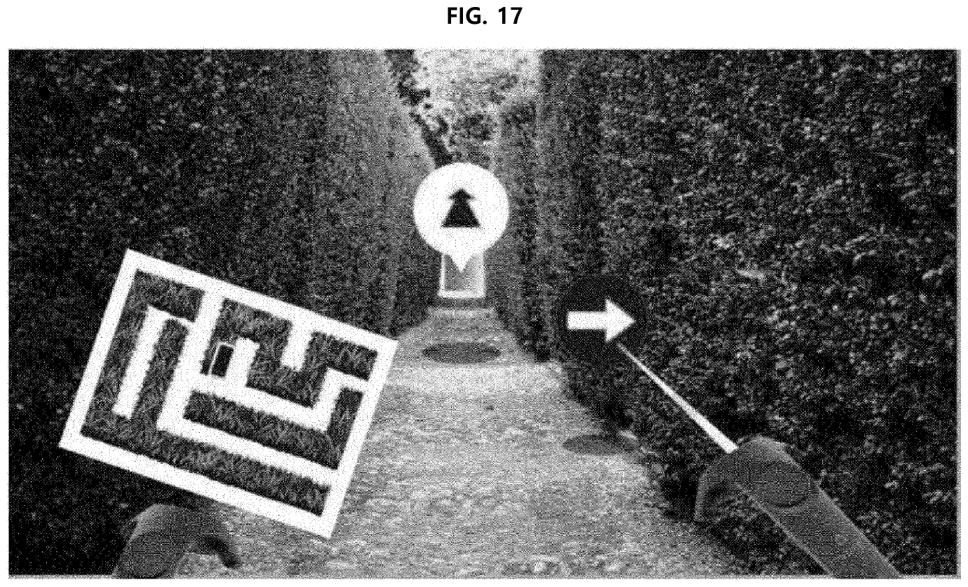

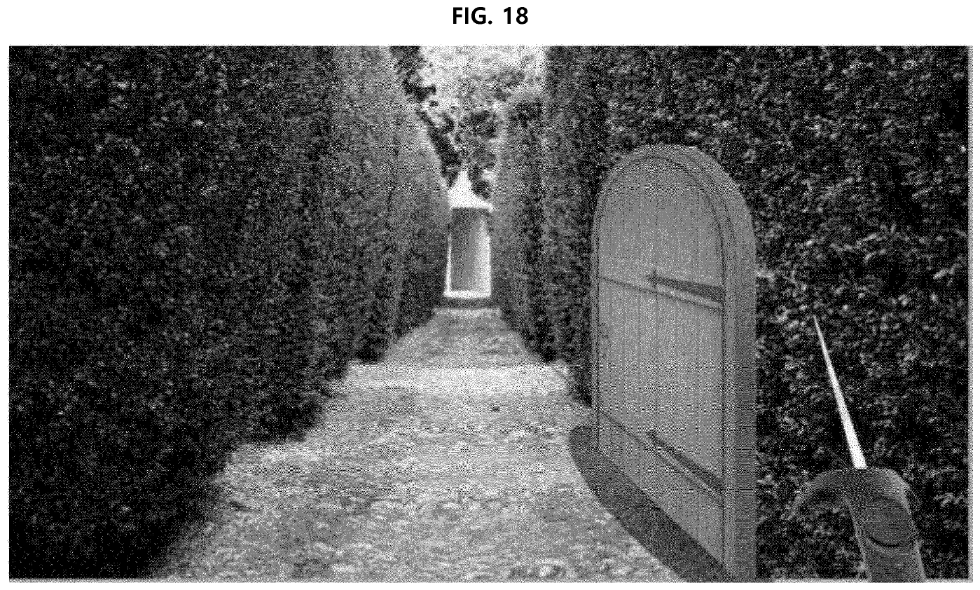

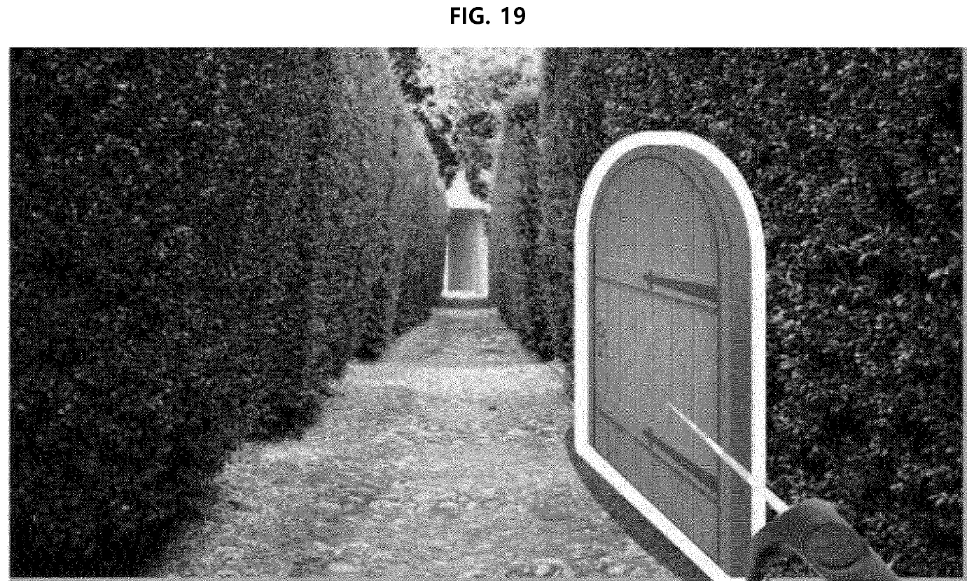

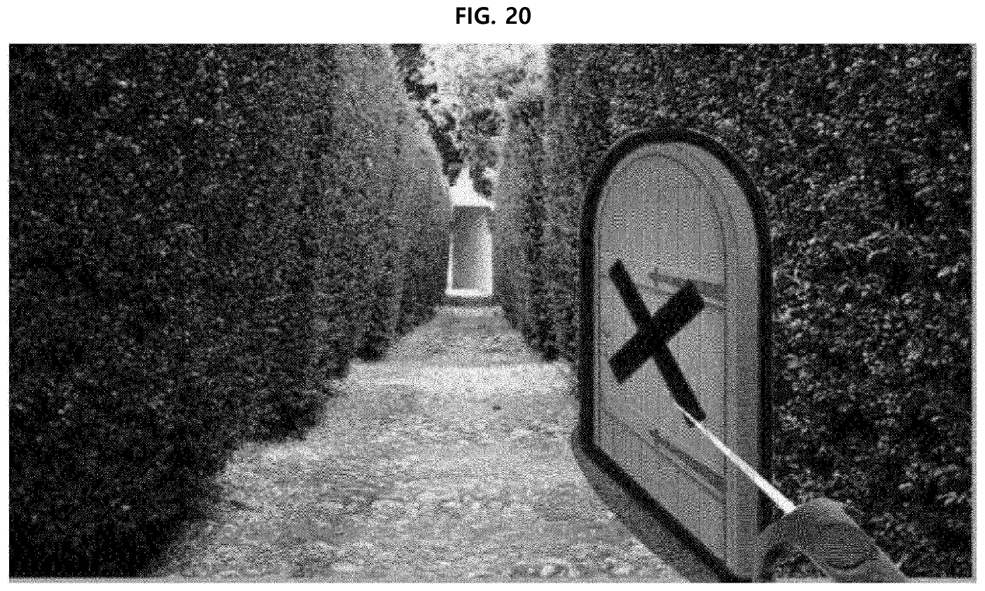

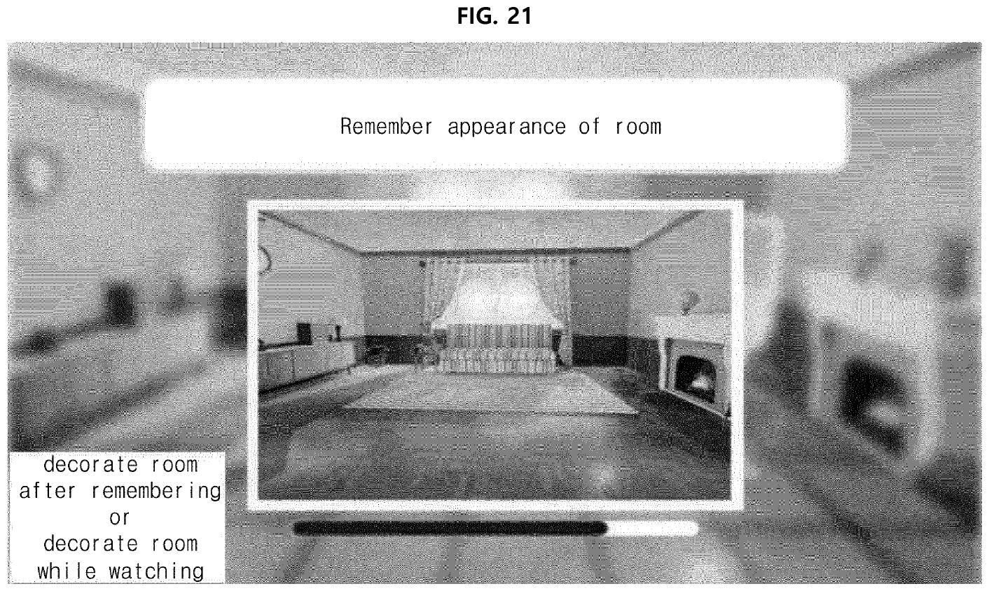

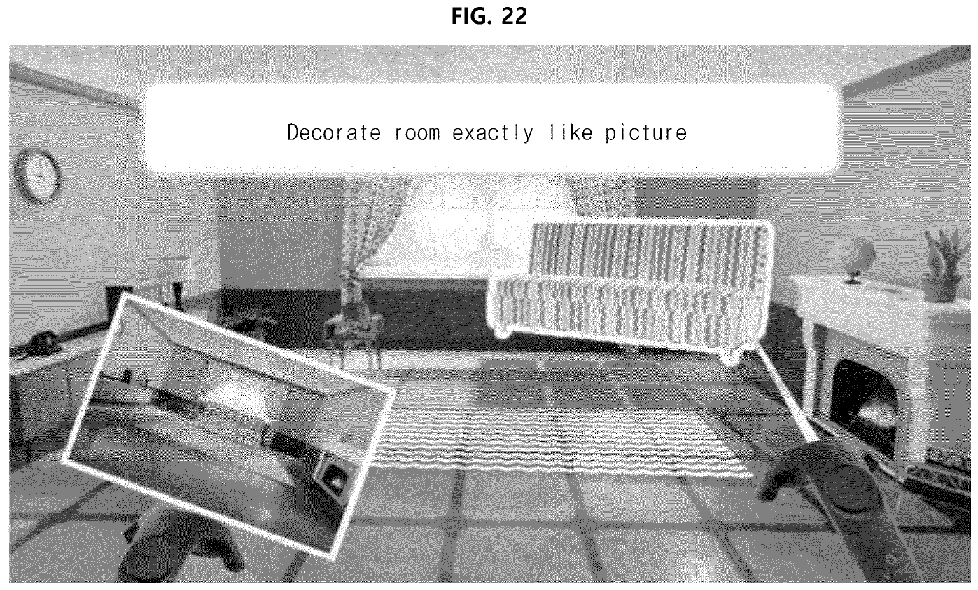

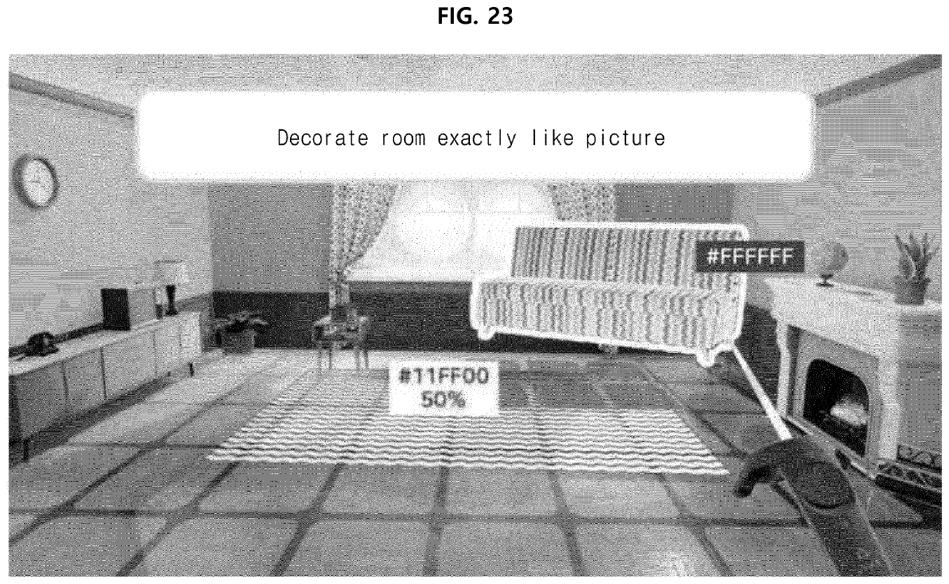

Hereinafter, a dementia patient training method of a dementia patient training system using VR will be described with reference to FIGS. 3 to 24.

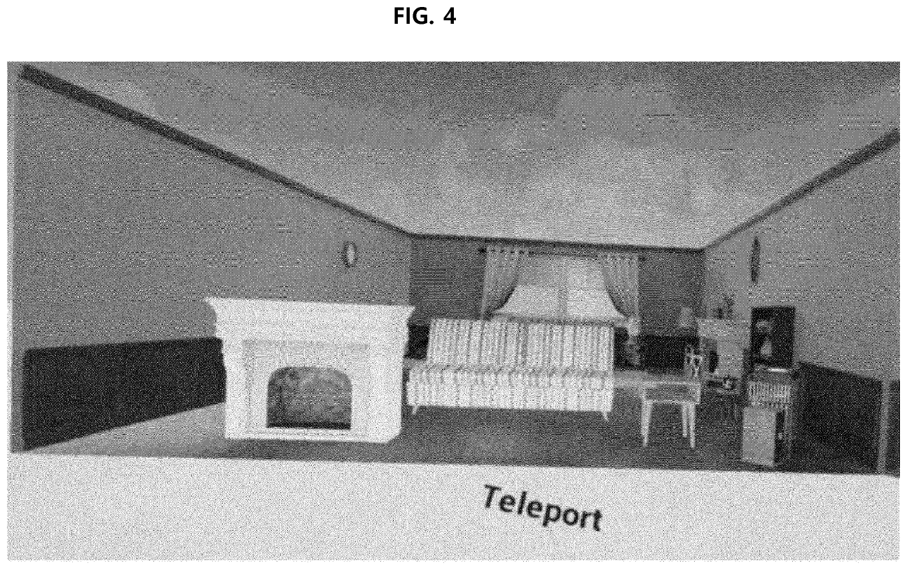

FIG. 3 is a flowchart of a dementia patient training method using VR, according to an embodiment of the present disclosure. FIGS. 4 to 23 are screens of a virtual environment illustrating a mission provided by a dementia patient training system using VR and a process in which a patient resolves the mission.

Above all, referring to FIG. 3, a dementia patient training method using VR according to an embodiment of the present disclosure may invite at least one friend user to a user's virtual environment, and may receive cognitive ability improvement mission content through collaboration so as to be resolved.

Accordingly, a first user may proceed with cognitive ability improvement mission content in a method including a login step S11, a mission selection step S21, a mission resolution step S31, a friend invitation step S41, and a logout step S51. A second user may proceed with cognitive ability improvement mission content through collaboration or competition, in a method including a login step S12, a friend invitation acceptance step S42, and a logout step S52.

Referring to FIGS. 1 to 3, in the login step (S11, S12), each of a plurality of users may access the server 100 through the terminal 200 of each of the plurality of users.

The respective user terminal 200 may provide each user with a login screen including an ID input window and a password input window through an output unit.

The login screen may provide a window for adding a friend, and thus the first user may invite a friend. (S41, S42)

Afterward, after the first user logs in, the first user may mount the output unit 300 so as to receive a virtual environment, in which a main screen is displayed.

A list screen may be provided on the main screen. Herein, the list screen may include a friend list and a mission-type content list.

Afterward, in the accessed virtual environment, the first user or the second user may receive the following missions and may perform and resolve presented missions through an input such as actions and selections associated with the missions. (S31)

Above all, referring to FIGS. 4 to 7, a dementia patient training system using VR according to an embodiment of the present disclosure may provide a mission for verifying VR adaptation ability and improving the VR adaptation ability.

The VR adaptation mission may include a mission for finding an incorrect object, a mission for selecting a moving object, and the like in two virtual spaces the same as each other.

Herein, missions having an appropriate difficulty level may be combined and presented depending on the cognitive ability determined by the cognitive ability determination module 140 of a user (patient).

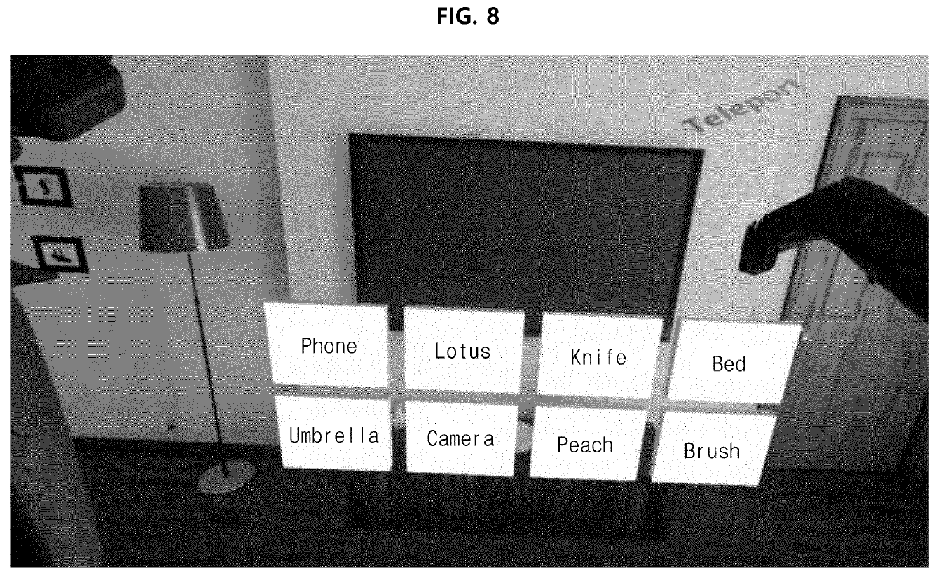

Afterward, referring to FIGS. 8 to 12, the dementia patient training system using VR according to an embodiment of the present disclosure may provide a mission for verifying a memory cognitive ability and improving the memory cognitive ability.

The memory cognition mission may include a mission for remembering a presented word and then selecting the presented word.

Herein, missions having an appropriate difficulty level may be combined and presented depending on the cognitive ability determined by the cognitive ability determination module 140 of the user (patient).

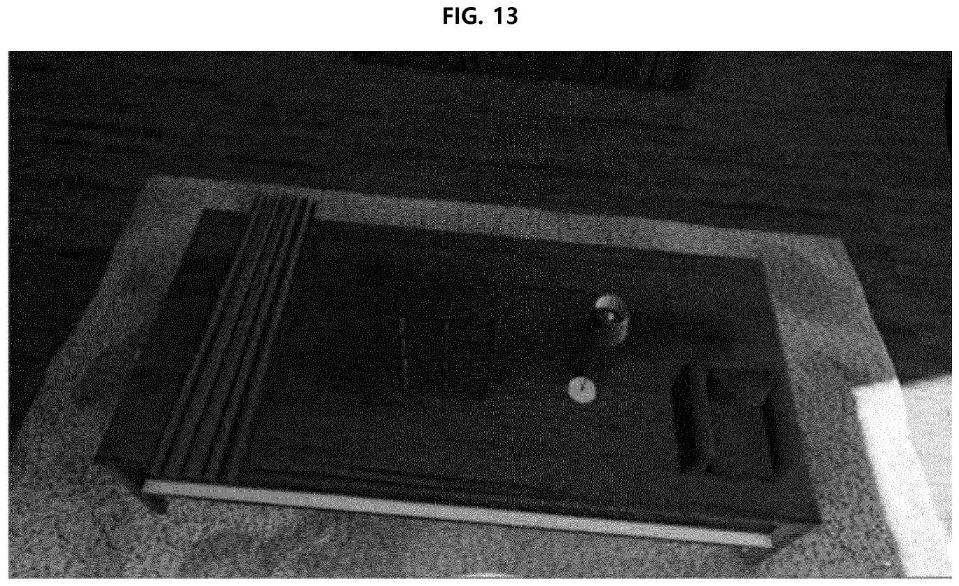

Afterward, referring to FIGS. 13 to 15, the dementia patient training system using VR according to an embodiment of the present disclosure may provide a mission for verifying a concentration cognitive ability and improving the concentration cognitive ability.

The concentration cognition mission may include a mission for selecting a presented object from among a plurality of listed objects or selecting other objects excluding the presented object.

Herein, missions having an appropriate difficulty level may be combined and presented depending on the cognitive ability determined by the cognitive ability determination module 140 of the user (patient).

Furthermore, although not illustrated, the dementia patient training system using VR according to an embodiment of the present disclosure may provide a mission for verifying a calculation cognitive ability and improving the calculation cognitive ability.

The calculation cognition mission may include a mission for counting the number of listed objects or calculating a calculation formula presented on a screen.

Herein, missions having an appropriate difficulty level may be combined and presented depending on the cognitive ability determined by the cognitive ability determination module 140 of the user (patient).

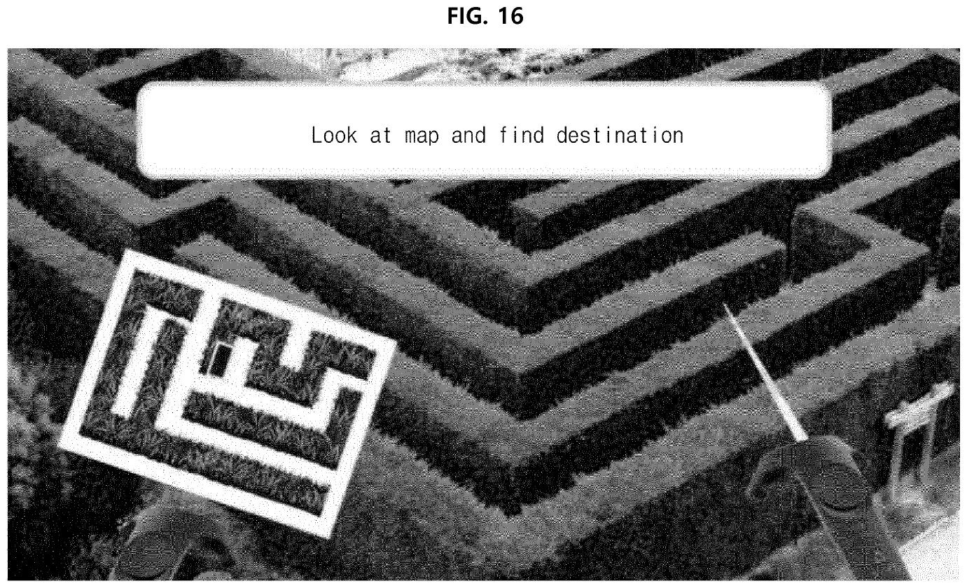

Afterward, referring to FIGS. 16 to 23, the dementia patient training system using VR according to an embodiment of the present disclosure may provide a mission for verifying a spatiotemporal cognitive ability and improving the spatiotemporal cognitive ability.

The space-time cognition mission may include a mission for escaping a maze presented in a virtual environment, a mission for placing furniture in a limited virtual space, and the like.

Herein, missions having an appropriate difficulty level may be combined and presented depending on the cognitive ability determined by the cognitive ability determination module 140 of the user (patient).

Furthermore, although not illustrated, the dementia patient training system using VR according to an embodiment of the present disclosure may provide a mission for verifying a thought processing cognitive ability and improving the thought processing cognitive ability.

The thought processing cognition mission may include a mission for verifying the user's processing ability in a presented situation.

Herein, missions having an appropriate difficulty level may be combined and presented depending on the cognitive ability determined by the cognitive ability determination module 140 of the user (patient).

Furthermore, although not illustrated, the dementia patient training system using VR according to an embodiment of the present disclosure may provide a mission for verifying a language cognitive ability and improving the language cognitive ability.

The language cognition mission may include a mission for identifying suggested phonemes, syllables, or words or identifying hidden syllables.

Herein, missions having an appropriate difficulty level may be combined and presented depending on the cognitive ability determined by the cognitive ability determination module 140 of the user (patient).

Meanwhile, each presented mission may not only determine a specific cognitive ability, but may also determine two or more cognitive abilities through a single mission.

For example, through the missions shown in FIGS. 8 to 12, it is possible to determine and improve a memory cognitive ability, a concentration cognitive ability, and a language cognitive ability.

Afterward, the first user and the second user may logout in a virtual environment by performing each logout step (S51, S52).

What has been described above is only one embodiment for implementing the dementia patient training system using VR according to an embodiment of the present disclosure. The present disclosure is not limited to an embodiment. As claimed in the following claims, those skilled in the field to which the present disclosure pertains will have the technical spirit of the present disclosure to the extent that various changes are capable of being implemented, without departing from the gist of the present disclosure.

1. A dementia patient training system using virtual reality (VR), which includes a server providing the VR to a dementia patient accessed over a network, the system comprising:
a process module configured to improve a virtual environment of the VR and to provide the patient with at least one mission content for improving a plurality of cognitive abilities in the virtual environment;
a storage module configured to store the mission content, selection data of the patient, motion data of the patient, and data about whether a mission of the patient is successful;
a communication module configured to communicate with a terminal of the patient over the network; and
a cognitive ability determination module configured to determine a cognitive ability of the patient through the selection data of the patient and the motion data of the patient for a mission provided by the process module,
wherein the communication module receives the selection data of the patient and the motion data of the patient, and
wherein the process module reflects the selection data of the patient and the motion data of the patient to the virtual environment.

2. The system of claim 1, wherein the mission content in the storage module is classified into a memory group for verifying a memory cognitive ability, a concentration group for verifying a concentration cognitive ability, a calculation ability group for verifying a calculation cognitive ability, a space-time group for verifying a spatiotemporal cognitive ability, a thought processing group for verifying a thought processing cognitive ability, and a language improvement group for verifying a language cognitive ability, and
wherein the process module selects at least one mission from each of the memory group, the concentration group, the calculation ability group, the space-time group, the thought processing group, and the language group so as to be provided to the patient.

3. The system of claim 2, wherein difficulty levels of a plurality of missions stored in each of the memory group, the concentration group, the calculation ability group, the space-time group, the thought processing group, and the language group in the storage module are set in a grade order.

4. The system of claim 3, wherein the process module selects a mission of a grade corresponding to the cognitive ability of the patient in each group, a mission of a grade immediately below the grade corresponding to the cognitive ability of the patient, and a mission of a grade immediately above the grade corresponding to the cognitive ability of the patient, which are 1:1:1, based on the cognitive ability of the patient for each group, which is classified at a previous try, so as to be provided to the patient at a next try,
wherein, when it is determined, based on the cognitive ability of the patient for the each group classified at the previous try, that the cognitive ability of the patient in a specific group is at the highest grade, the process module selects the mission of the grade corresponding to the cognitive ability of the patient and the mission of the grade immediately below the grade corresponding to the cognitive ability of the patient, which are 2:1, so as to be provided to the patient at the next try, and
wherein, when it is determined, based on the cognitive ability of the patient for the each group classified at the previous try, that the cognitive ability of the patient in a specific group is at the lowest grade, the process module selects the mission of the grade corresponding to the cognitive ability of the patient and the mission of the grade immediately above the grade corresponding to the cognitive ability of the patient, which are 2:1, so as to be provided to the patient at the next try.

5. The system of claim 4, wherein the cognitive ability determination module stores the cognitive ability of the patient for the each group, which is classified at each repeated try, in the storage module and determines whether the cognitive ability of the patient is improved or declined, based on information about the cognitive ability of the patient that accumulated at the each repeated try.

6. The system of claim 5, wherein the process module increases a time distribution amount within a predetermined time for a specific group, in which a cognitive ability declines, and decreases a time distribution amount within a predetermined time for a specific group in which a cognitive ability is improved, based on the cognitive ability of the patient for the each group classified at the previous try.

7. The system of claim 5, wherein the communication module transmits the information about whether the cognitive ability of the patient in the the each group is improved or declined, to the outside at a predetermined period or at an external request.

8. The system of claim 1, wherein a plurality of patients are capable of accessing the server through terminals of the plurality of patients,
wherein the plurality of patients include a first patient and a second patient, and
wherein the process module provides the plurality of patients with mission content in which the first patient and the second patient collaborate or compete with each other.

9. The system of claim 8, wherein the second patient accesses the server through a terminal of the second patient, and
wherein, when the second patient accepts a friend invitation event of the first patient in a virtual environment of the second patient, the process module receives a virtual environment of the first patient.
